(19) United States
(12) Patent Application Publication
(10) Pub. No.: US 2013/0142895 A1
(43) Pub. Date: Jun. 6, 2013

(54) IMMUNOSUPPRESSANT EXTRACTS DERIVED FROM MILLETTIA LAURENTII OR PSEUDOTSUGA PINACEAE

(71) Applicant: Harout Dersimonian, Kittery, ME (US)

(72) Inventor: Harout Dersimonian, Kittery, ME (US)

(73) Assignee: NatCure Sciences, Inc., Kittery, ME (US)

(21) Appl. No.: 13/582,496

(22) PCT Filed: Mar. 1, 2011
(86) PCT No.: PCT/US11/26627

§ 371 Date: Nov. 12, 2012

Related U.S. Application Data
(60) Provisional application No. 61/309,340, filed on Mar. 1, 2010.

(51) Int. Cl. A61K 36/486; A61K 36/15

(52) U.S. Cl. CPC A61K 36/486; A61K 36/15
USPC 424/770; 424/725

(57) ABSTRACT

One aspect of the present invention generally relates to deriving immunosuppressant extracts from Millettia laurentii or Pseudotsuga pinaceae. In addition, the present invention relates to methods of using immunosuppressant extracts of Millettia laurentii or Pseudotsuga pinaceae to treat an autoimmune condition, transplant rejection, or cancer.

### RELATED APPLICATIONS

This application claims the benefit of priority to U.S. Provisional Patent Application Ser. No. 61/309,340, filed Mar. 1, 2010.

### BACKGROUND OF THE INVENTION

Millettia laurentii, commonly known as Wenge or Awong, is a member of the Leguminosae Family of trees. Millettia laurentii grows in swampy areas of western Africa, including the countries of Cameroon, Gaboon, and the Democratic Republic of the Congo. Millettia laurentii is a dense wood used in carpentry and wood working.

When sanded, Millettia laurentii gives off a fine dust that can be irritating to skin, eyes, and lungs. Dr. DerSimonian observed that human contact with wood chips or splinters from Millettia laurentii frequently resulted in infection. Dr. DerSimonian has found that this effect is in sharp contrast to other natural wood products used in carpentry or wood working.

There is a need in the industry to understand the medicinal effects of Millettia laurentii.

### SUMMARY OF THE INVENTION

The present invention generally relates to deriving immunosuppressant extracts from Millettia laurentii or Pseudotsuga pinaceae. In addition, the present invention relates to methods of using immunosuppressant extracts of Millettia laurentii or Pseudotsuga pinaceae to treat autoimmune conditions, transplant rejection, and cancer.

### BRIEF DESCRIPTION OF THE FIGURES

FIG. 1 is a bar chart illustrating inhibition of lymphocyte proliferation in a mixed lymphocyte reaction (MLR) assay using human PBMC donor A and donor B, alone or in mixed combination;

FIG. 2 is a bar chart illustrating that production of inflammatory cytokines IL-2, IFNγ, TNFα, and GMCSF was inhibited in the human MLR assay.

FIG. 3 is a bar chart illustrating that production of two additional inflammatory cytokines, IL-6 and IL-8, was also inhibited in the human MLR assay.

### DETAILED DESCRIPTION OF THE INVENTION

The invention meets a need in the art by providing water soluble extracts derived from Millettia laurentii or Pseudotsuga pinaceae, having immunosuppressant activity to treat autoimmunity, transplant rejection, and cancer. Some of the practical applications of this invention are to: 1) treat any and all autoimmune diseases; 2) treat transplant patients in order to avoid organ, tissue, or cell rejection; and 3) treat cancer patients in conjunction with standard chemotherapy to target the residual cancer stem cells that divide very slowly or are arrested.

Millettia laurentii has a potent immunosuppressive effect on inflammation and lymphocyte activation and may also target non-dividing cell types, including cancer stem cells. The product is a naturally occurring molecule or group of them that can be extracted by incubating finely sanded wood or bark dust in standard tissue culture media, saline, or water, or an organic solution.

Millettia laurentii is a dense wood, which, when sanded, gives off a fine dust that can be irritating to skin, eyes, and lungs. Direct observations show that human contact with wood chips or splinters frequently develop into infections. Dr. DerSimonian made the original observation that a local wood worker was getting more infections due to splinters from one particular type of exotic wood than from other types. The exotic woods that produced infections in wood workers due to splinters include those listed in Table 1.

TABLE 1
Exotic Woods that Produced Infections Due to Splinters.

| | | |
|---|---|---|
| Afrormosia | Eye, skin irritant; effects on nervous system; | dust, |
| | rhinitis; asthma; splinters go septic. | wood |
| Douglas Fir | Nasal cancer; rhinitis; respiratory | dust, |
| | problems; dermatitis; splinters go septic | wood |
| Greenheart | Cardiac and intestinal disorder; severe | dust, |
| | throat irritant; sensitizer; splinters go septic | wood |
| Mansonia | Nosebleeds; sneezing; skin irritant; | dust, |
| | asthma; respiratory problems; cardiac disorders; | wood, |
| | splinters go septic; bark highly toxic | bark |
| Wenge | Eye, skin, respiratory system irritant; | dust, |
| | giddiness; drowsiness; visual problems; stomach | wood |
| | cramps; sensitizer; splinters go septic | |

After investigating whether any other type of wood splinter caused such a reaction, Dr. DerSimonian came up with a hypothesis: that such an exotic wood may have either an infectious agent in the product or the product had a potent anti-inflammatory, immunosuppressant, or anti-innate immune activity (or activities). Finding no infectious agent in the wood, the anti-inflammatory, immunosuppressant, or anti-innate immune activity (or activities) was tested in the laboratory using a standard immune assay of the water soluble fraction of the natural wood product. A potent inhibitory activity was identified in this water soluble fraction. Further testing is underway to isolate and identify the natural product activity using biochemical approaches.

The initial assay indicated a potent inhibitory activity on human lymphocytes. It is not known if this is a direct effect on the lymphocytes or other cell types in the human blood. Crucial inflammatory cytokines produced in an immune response were also inhibited. Thus, the water soluble fraction made from the natural product was tested and demonstrated its anti-inflammatory, immunosuppressant, or anti-innate immune activity (or activities) or potency in a dose dependent manner (see Protocol and Results, infra). This potent activity is presumed to be due to one or more novel constituents of the extract,

In one embodiment, the invention relates to the identification of a novel immunosuppressant compound that is water soluble and extracted from the exotic wood Millettia laurentii from the Leguminosae tree Family. In another embodiment, the novel immunosuppressant compound that is water soluble may be extracted from a very closely related natural product with the common name of Panga-panga that grows in the open forests of Mozambique and Tanzania or the plant Pseudotsuga pinaceae with the common name Douglas fir, which grows in the northwestern region of North America.

In some embodiments, the invention provides for an extract derived from Millettia laurentii or Pseudotsuga pinaceae for treating autoimmunity, transplant rejection, and cancer, prepared according to the method comprising the steps of collecting one gram of wood dust from Millettia laurentii or Pseudotsuga pinaceae, suspending the one gram of wood dust in RPMI media containing 10% human AB serum to create a suspension solution, storing the suspension solution at 4° C. for 48 hours, passing the suspension solution through a 0.2 μm filter to sterilize the solution and collect the water-soluble fraction, adding 100 μL of the water-soluble fraction to a final volume of 200 uL to make 1× factor, or alternatively, 20 μL of soluble factor to a final volume of 200 uL to make ⅕× factor concentration.

In other embodiments, the invention provides for methods of treating autoimmune conditions, transplant rejection, and cancer, comprising the step of administering to a subject in need thereof a therapeutically effective amount of a water soluble extract of Millettia laurentii or Pseudotsuga pinaceae.

In some embodiments, the therapeutically effective dose may be administered by a variety of routes, including, but not limited to, being administered orally, parenterally, by injection, subcutaneously, intravenously, intramuscularly, intraperitoneally, intraarterially, intralesional, epidurally, intrathecally, intracerebrally, topically, intranasally, locally (e.g., surgical application or surgical suppository), sublingually, submucosally, rectally, vaginally, transdermally, or by pulmonary route (e.g., aerosols, inhalation, or powder).

In some embodiments, the extract of Millettia laurentii or Pseudotsuga pinaceae may be administered continuously by infusion, and in other embodiments, by bolus injection. Such compositions are prepared in a manner well known in the pharmaceutical arts.

In some embodiments, the therapeutically effective dose administered may be from 1 ng to 1 g. Administration may be topical for a skin remedy, or systemic for an internal remedy.

In some embodiments the therapeutically effective amount of the extract of Millettia laurentii or Pseudotsuga pinaceae is administered from 1 to 10 times a day. In other embodiments, the therapeutically effective amount of the extract of Millettia laurentii or Pseudotsuga pinaceae is administered from 1 to 10 times every other day. In other embodiments, the therapeutically effective amount of the extract of Millettia laurentii or Pseudotsuga pinaceae is administered from 1 to 10 times every week. In other embodiments, the therapeutically effective amount of the extract of Millettia laurentii or Pseudotsuga pinaceae is administered from 1 to 10 times every month.

In some embodiments the therapeutically effective amount of the extract of Millettia laurentii or Pseudotsuga pinaceae is administered daily. In other embodiments, the therapeutically effective amount of the extract of Millettia laurentii or Pseudotsuga pinaceae is administered from 2 to 10 times each day. In other embodiments, the therapeutically effective amount of the extract of Millettia laurentii or Pseudotsuga pinaceae is administered from 2, 3, 4, 5, 6, 7, 8, 9, or 10 times each day.

In some embodiments, the autoimmune disorder treated is Addison's disease, amyotrophic lateral sclerosis, asthma, atopic allergy, atopic dermatitis, Celiac disease, chronic fatigue syndrome, colitis, Crohn's disease, Cushing's syndrome, dermatitis, type-1 diabetes, type-2 diabetes, Dressler's syndrome, eczema, fibromyalgia, gastritis, Grave's disease, Guillian-Barre syndrome, hepatitis, irritable bowel syndrome, lupus, lyme disease, Meniere's disease, multiple sclerosis, myositis, polymyositis, psoriasis, rheumatoid arthritis, sarcoidosis, scleroderma, or ulcerative colitis.

In other embodiments, the transplant rejection treated is associated with transplant of a heart, intestine, kidney, liver, lung, pancreas, thymus, muscle, skin, blood, bone marrow, immune cells, or stem cells.

In other embodiments, the cancer treated is bladder cancer, bone cancer, brain cancer, breast cancer, cervical cancer, colon cancer, esophageal cancer, eye cancer, heart cancer, kidney cancer, laryngeal cancer, leukemia, lung cancer, mouth cancer, nasal cancer, oral cancer, pancreatic cancer, prostate cancer, rectal cancer, skin cancer, testicular cancer, throat cancer, thyroid cancer, or vaginal cancer.

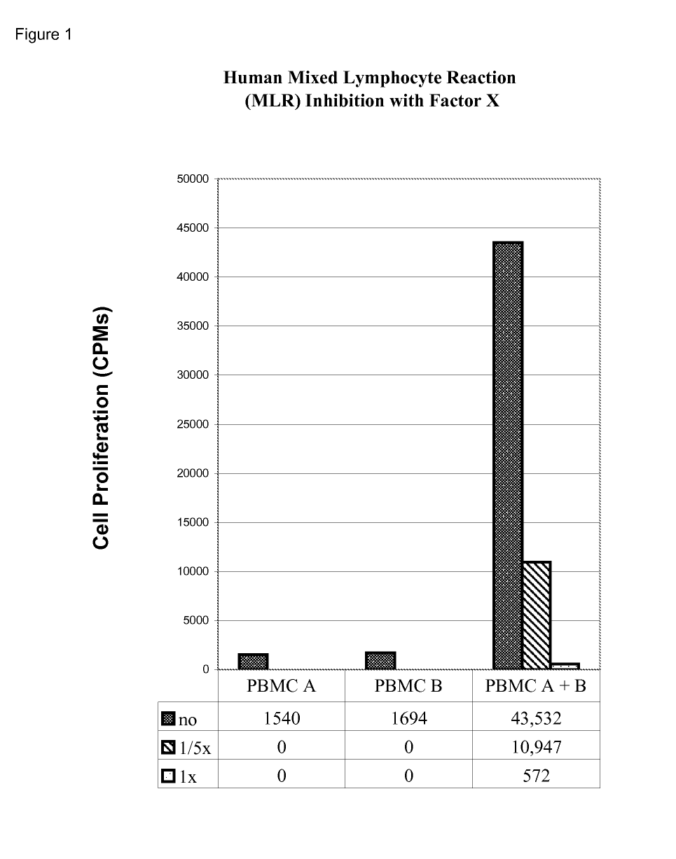

FIG. 1 is a bar chart depicting inhibition of lymphocyte proliferation in mixed lymphocyte reaction (MLR) assay using human PBMC donor A and donor B. PBMC alone or in mixed combination were incubated for 5 days at 37° C. in CO₂ before ³H-thymidine was added to measure thymidine incorporation (CPM; counts per minute) and proliferation. Lymphocyte proliferation of normal PBMC donor A alone (left column) or PBMC donor B alone (middle column) were included for background control. Only when PBMC from donor A and B were mixed, was a strong proliferation demonstrated (right columns) in the absence of any Factor X (black bar). This MLR response was inhibited more than 75% with ⅕ dilution (grey bar) and completely with undiluted Factor X (white column).

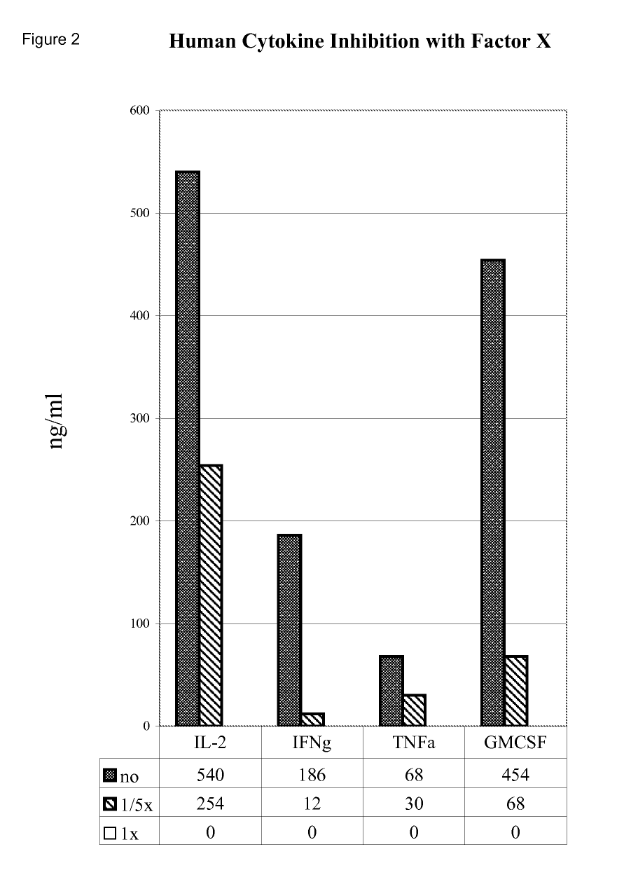

FIG. 2 is a bar chart depicting production of inflammatory cytokines IL-2, IFNγ, TNFα, and GMCSF were inhibited in the human MLR assay. Cytokine productions [ng/ml] were detected in the MLR assay supernatant in the absence of the inhibitor Factor X (black columns), and in significantly decreased levels in the presence of ⅕ diluted Factor X (grey columns), and decreased to undetectable levels with undiluted Factor X (white columns). The complete inhibition of these inflammatory cytokines underscores the potency of this natural product.

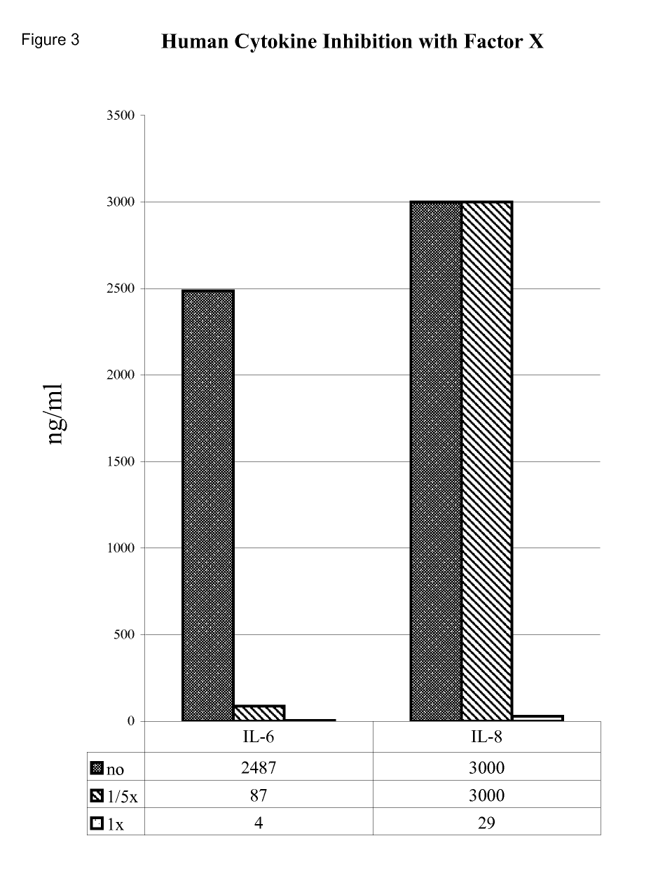

FIG. 3 is a bar chart depicting that production of inflammatory cytokines IL-6 and IL-8 were also inhibited in the human MLR assay. Cytokine production was detected in the MLR without the inhibitor (black columns). Significant blocking was seen for IL-6 when diluted (⅕×) inhibitor factor was present (grey columns), but not for IL-8. Complete inhibition was seen when undiluted Factor X was added to the assay (undetected, white columns).

Definitions

As used herein, unless otherwise defined, the singular forms “a”, “an”, and “the” include plural references unless the context clearly dictates otherwise.

As used herein, unless otherwise defined, the terms “Millettia laurentii,” “Panga-panga,” “Afrormosia,” “Douglas Fir,” “Pseudotsuga pinaceae,” “Greenheart,” “Mansonia,” “Wenge,” “Awong,” or “exotic wood” includes heartwood, bark, seeds, leaves, branches, and roots from these tree families.

As used herein, unless otherwise defined, the term “therapeutically effective amount” of the water soluble extract of Millettia laurentii or Pseudotsuga pinaceae is one which reduces the problems usually associated with treating autoimmunity, transplant rejection, and cancer.

Protocol and Results

Water-soluble component of a natural product was demonstrated to contain potent anti-inflammatory, immunosuppressant, or anti-innate immune activity (or activities). Experiments were designed to evaluate the effect and potency of extract from a natural product that cause splinter infection in the skin of healthy individuals where most other related products tested do not. Here, we used the standard MLR assay to assess whether components from this natural product had anti-inflammatory, immunosuppressant, or anti-innate immune activity (or activities) effect. Human mixed lymphocyte reaction is a good way to measure lymphocyte activation and proliferation as well as other factors that may inhibit or enhance the response.

Human PBMC immune response in vitro was blocked with the water-soluble material. FIG. 1 shows the background response of PBMC A alone in the first column (1540 cpm), while the second column shows the background response (1694 cpm) for the second PBMC alone. In contrast, mixed PBMC showed a strong standard proliferative response (43,532 cpm) as seen in third column. Moreover, when water soluble material was added to this mixed PBMC during the assay, potent inhibition was detected. When ⅕× material was added, the response was reduced more than 75% from 43,532 to 10,947 cpm. And when higher concentration of material (1× factor) was added, the response was inhibited to background levels. The immune response was completely blocked from 43,532 to 572 cpm.

Another way to measure immune response or inhibition of that response is to measure the production of the inflammatory cytokines that are produced during MLR. We tested the effect of the natural product on the production of cytokines and found a potent inhibition in the production of a number of the inflammatory cytokines. For example, IL-2 production in the MLR assay decreased significantly from 540 ng/ml to 254 ng/mL when ⅕× factor was added and to undetectable levels (0 ng/ml) when 1× factor was added to the assay (FIG. 2; first group column). Likewise, IFNγ, TNFα, and GMCSF production were undetectable (FIG. 2; white columns*) when 1× factor was added to the wells. Significant blocking was also seen when only ⅕× factor was added to the assay (FIG. 2; grey columns).

Both IL-6 and IL-8 cytokine production were also effected (FIG. 3). While inhibition of IL-6 was readily detected from 2487 ng/ml to 87 ng/mL with the ⅕× factor and to undetectable levels at the 1× factor levels, the IL-8 levels seemed saturated where no inhibition was detected with ⅕× factor, but again completely inhibited with the natural product at the 1× concentration (FIG. 3, white columns).

These results show that proliferation can be blocked significantly when ⅕× concentration of the natural product was used and this inhibition was more complete when 1× concentration of the water soluble material was tested. This was true also in the inhibition of inflammatory cytokine productions as indicated by the decreased IL-2, IL-6, IL-8, IFNγ, TNFα, and GMCSF.

Human MLR Proliferation Assay

Peripheral blood was collected from healthy unrelated donors. Human peripheral blood mononuclear cells (PBMC) were isolated by Ficoll-Pague gradient (Yamada, K., D. H. Sachs, H. DerSimonian. 1995. J. Immunol. 155:5249) and plated in 96-well flat-bottom microtiter plates. Cells were plated at 2×10⁵ cells/well alone or in mixed cultures (PBMC A+PBMC B). Mixed PBMC cultures were also incubated with medium alone (no factor), with ⅕× concentration of water-soluble natural product extract (⅕ factor x), or with 1× concentration of water-soluble natural product extract (1× factor) at 37° C. in CO2 incubator. All experiments were done in RPMI medium (Life Technologies, Grand Island, N.Y.) supplemented with 10% human AB serum (HyClone, Logan, Utah). Supernatants were harvested from appropriate cultures at indicated times and used for cytokine detection. To determine the proliferative response, cells were pulsed with [³H]thymidine (1 μCi/well) for 20 h and harvested with a cell harvester (Packard Instrument, Meriden, Conn.) at the indicated time points. Thymidine incorporation was determined by counting the filter plate using a microplate scintillation counter (model B9906; Packard Instrument).

Natural Product Water-Soluble Material

Roughly one gram of wood dust from the natural product was suspended into 20 mL of RPMI containing 10% human AB Serum. The suspended material was stored at 4° C. for 48 hours to release activity into the ‘water-soluble’ fraction. Suspended material was passed through 0.2 μm filter to sterilize and collect the ‘water-soluble’ fraction. Soluble material was added to the MLR assay as indicated above. Remaining supernatant was stored at 4° C. for future use.

One hundred (100) μL of soluble factor added to a final volume of 200 μL to make 1× factor and 20 μL of soluble factor added to a final volume of 200 μL to make ⅕× factor concentration.

Discussion of Results

In these studies, we have shown that a material activity from a natural product was able to block proliferation of human cells in an in vitro mixed culture. Even with diluted concentration of the material (⅕× factor), proliferation of the human cells in a primary response was inhibited significantly and completely with the undiluted (1× factor) material. Interestingly, this material activity is found to be in the water soluble fraction. Although the identity of the material is yet not known, it seems highly likely that it is hydrophilic and easily soluble in water. This may be an important characteristic in developing therapeutic reagents for autoimmunity, transplantation or cancer treatments.

These initial findings help explain the potent activity seen in vivo where infections are common. It is interesting to comment on the mechanism of arresting the activation for the immune cells—since they do appear to be arrested before proliferation. For this reason, it is of interest to check the effect of this material on cancer cell types, especially when one considers the characteristics of cancer stem cells that do not readily proliferate during chemotherapy treatment and, therefore, cannot be depleted during conventional chemotherapy treatment.

The foregoing description of the embodiments of this invention has been presented for purposes of illustration and description. It is not intended to be exhaustive or to limit the invention to the precise form disclosed, and obviously, many modifications and variations are possible. Such modifications and variations that may be apparent to a person skilled in the art are intended to be included within the scope of this invention as defined by the accompanying claims.

We claim:

1. An extract from Millettia laurentii or Pseudotsuga pinaceae prepared according to a method comprising the steps of:
combining a sample of wood from Millettia laurentii or Pseudotsuga pinaceae and an aqueous solution, thereby forming a mixture,
passing the mixture through a filter, thereby forming an extract and a filter cake, and
collecting the extract.

2. The extract of claim 1, wherein the sample of wood from Millettia laurentii or Pseudotsuga pinaceae is collected from heartwood, bark, seeds, leaves, branches, or roots.

3. The extract of claim 1, wherein the aqueous solution is water or cell-culture medium.

4. The extract of claim 3, wherein the cell-culture medium contains serum.

5. The extract of claim 1, wherein the filter has a pore size of about 0.2 μm.

6. A method for treating an autoimmune disorder, comprising the step of administering to a subject in need thereof a therapeutically effective amount of an extract of Millettia laurentii or Pseudotsuga pinaceae.

7. The method of claim 6, wherein the autoimmune disorder is: Addison's disease, amyotrophic lateral sclerosis, asthma, atopic allergy, atopic dermatitis, Celiac disease, chronic fatigue syndrome, colitis, Crohn's disease, Cushing's syndrome, dermatitis, type-1 diabetes, type-2 diabetes, Dressler's syndrome, eczema, fibromyalgia, gastritis, Grave's disease, Guillian-Barre syndrome, hepatitis, irritable bowel syndrome, lupus, lyme disease, Meniere's disease, multiple sclerosis, myositis, polymyositis, psoriasis, rheumatoid arthritis, sarcoidosis, scleroderma, or ulcerative colitis.

8. The method of claim 6, wherein the extract of Millettia laurentii or Pseudotsuga pinaceae is administered orally, topically, transmucosally, by inhalation, or by injection.

9. The method of claim 8, wherein the extract of Millettia laurentii or Pseudotsuga pinaceae is administered by injection, and the injection is intraarterial, intracerebral, intradermal, intralesional, intramuscular, intraperitoneal, intravenous, intrathecal, or subcutaneous.

10. The method of claim 6, wherein the therapeutically effective amount of the extract of Millettia laurentii or Pseudotsuga pinaceae is from 1 ng to 1 g.

11. The method of claim 6, wherein the therapeutically effective amount of the extract of Millettia laurentii or Pseudotsuga pinaceae is administered daily.

12. A method for treating transplant rejection, comprising the step of administering to a subject in need thereof a therapeutically effective amount of an extract of Millettia laurentii or Pseudotsuga pinaceae.

13. The method of claim 12, wherein the transplant rejection is associated with transplant of a heart, intestine, kidney, liver, lung, pancreas, thymus, muscle, skin, blood, bone marrow, immune cells, or stem cells.

14. The method of claim 12, wherein the extract of Millettia laurentii or Pseudotsuga pinaceae is administered orally, topically, transmucosally, by inhalation, or by injection.

15. The method of claim 14, wherein the extract of Millettia laurentii or Pseudotsuga pinaceae is administered by injection, the injection is intraarterial, intracerebral, intradermal, intralesional, intramuscular, intraperitoneal, intravenous, intrathecal, or subcutaneous.

16. The method of claim 12 wherein the therapeutically effective amount of the extract of Millettia laurentii or Pseudotsuga pinaceae is from 1 ng to 1 g.

17. The method of claim 12, wherein the therapeutically effective amount of the extract of Millettia laurentii or Pseudotsuga pinaceae is administered daily.

18. A method for treating cancer, comprising the step of administering to a subject in need thereof a therapeutically effective amount of an extract of Millettia laurentii or Pseudotsuga pinaceae.

19. The method of claim 18, wherein the cancer is bladder cancer, bone cancer, brain cancer, breast cancer, cervical cancer, colon cancer, esophageal cancer, eye cancer, heart cancer, kidney cancer, laryngeal cancer, leukemia, lung cancer, mouth cancer, nasal cancer, oral cancer, pancreatic cancer, prostate cancer, rectal cancer, skin cancer, testicular cancer, throat cancer, thyroid cancer, or vaginal cancer.

20. The method of claim 18, wherein the extract of Millettia laurentii or Pseudotsuga pinaceae is administered orally, topically, transmucosally, by inhalation, or by injection.

21. The method of claim 20, wherein the extract of Millettia laurentii or Pseudotsuga pinaceae is administered by injection, the injection is intraarterial, intracerebral, intradermal, intralesional, intramuscular, intraperitoneal, intravenous, intrathecal, or subcutaneous.

22. The method of claim 18 wherein the therapeutically effective amount of the extract of Millettia laurentii or Pseudotsuga pinaceae is from 1 ng to 1 g.

23. The method of claim 18, wherein the therapeutically effective amount of the extract of Millettia laurentii or Pseudotsuga pinaceae is administered daily.
